(19) United States
(12) Patent Application Publication
(10) Pub. No.: US 2025/0152916 A1
(43) Pub. Date: May 15, 2025

(54) CATHETER FOR ADMINISTERING A LOCAL ANESTHETIC

(71) Applicant: B. Braun Melsungen AG, Melsungen (DE)

(72) Inventor: Jens Wildhagen, Hannover (DE)

(21) Appl. No.: 18/945,693

(22) Filed: Nov. 13, 2024

(30) Foreign Application Priority Data
Nov. 14, 2023 (DE) 10 2023 131 697.4

(51) Int. Cl. A61M 25/00; A61M 19/00; A61M 25/02; A61M 25/06

(52) U.S. Cl. CPC A61M 25/0054; A61M 19/00; A61M 25/0023; A61M 25/0069; A61M 25/02; A61M 25/0662; A61M 2025/0681; A61M 2205/0216; A61M 2205/0233; A61M 2205/025; A61M 2205/054; A61M 2209/088

(57) ABSTRACT

A catheter for administering a local anaesthetic has a catheter shaft extending between a proximal end and a distal end, a catheter hub arranged at the proximal end, and a catheter tip arranged at the distal end. The catheter shaft has a stretch section configured to be easily stretchable in such a way that, in the event of a proximal tensile loading of the catheter hub, an at least partial stretching of the catheter shaft takes place, as a result of which a proximal dislocation of the catheter tip is avoided.

### CROSS-REFERENCE TO RELATED APPLICATIONS

This application claims priority under 35 U.S.C. § 119 to German Application No. 10 2023 131 697.4, filed on Nov. 14, 2023, the content of which is incorporated by reference herein in its entirety.

### FIELD

The present disclosure relates to a catheter for administering a local anaesthetic, the catheter having a catheter shaft, which extends between a proximal end and a distal end, a catheter tip, which is arranged at the distal end, and a catheter hub, which is arranged at the proximal end.

### BACKGROUND

Catheters are generally known in the field of medical engineering and are provided for use in pain therapy. In a pain therapy called peripheral nerve block (PNB), the catheter is advanced distally with the aid of an insertion aid until the catheter tip is placed directly on the nerve that is to be anaesthetized. Cannulas and/or capillaries are used as insertion aids, it being possible in principle to make a distinction between different attachment techniques (“cannula over the needle”, “cannula through the needle”, “cannula through the capillary”). To anaesthetize the nerve, the local anaesthetic is dispensed via one or more catheter outlets located in the region of the catheter tip.

### SUMMARY

It is an object of the present disclosure to provide a catheter of the type mentioned at the outset that allows improved pain therapy.

This object is achieved by the fact that the catheter shaft has a stretch section which is configured to be easily stretchable in such a way that, in the event of proximal tensile loading of the catheter hub, an at least partial stretching of the catheter shaft takes place, as a result of which a proximal dislocation of the catheter tip is avoided. The present disclosure is based on the finding that unintentional withdrawal of the catheter tip (proximal dislocation) can lead to insufficient flushing of the nerve with the local anaesthetic and can thus adversely affect the efficacy of the pain therapy. Such dislocation of the catheter tip is avoided by the solution according to the present disclosure. For this purpose, the catheter shaft comprises said stretch section. The stretch section is configured to be easily stretchable. Easily stretchable means that the stretch section has a comparatively low tensile strength and/or a comparatively high tensile compliance and is therefore easily stretchable, i.e. extensible in its length. In the event of proximal tensile loading of the catheter hub and thus of the catheter shaft, which may be caused, for example, by mobilization of the patient, the stretch section is stretched, i.e. lengthened and/or extended. On account of this increase in length of the catheter shaft in the region of the stretch section, there is a correspondingly reduced dislocation or, in the best case, no proximal dislocation of the catheter tip. In one embodiment, the stretch section is formed for elastic stretching. In a further embodiment, the stretch section is formed for plastic stretching. In a further embodiment, the stretch section is formed for clastic-plastic stretching. In one embodiment, the comparatively easy stretchability of the stretch section is achieved through a corresponding choice of material for the stretch section. In a further embodiment, the stretch section alternatively or additionally has an easily stretchable design, for example an accordion-shaped or spiral-shaped design. Preferably, the catheter hub is configured for releasable connection to an insertion aid, and the insertion aid can be designed in particular as a capillary or as a cannula.

In an embodiment of the present disclosure, the stretch section has a tensile compliance of at least 10 mm/N, preferably of at least 100 mm/N, particularly preferably of at least 1000 mm/N. The abovementioned ranges of values for the tensile compliance of the stretch section have proven to be particularly advantageous for avoiding said proximal dislocation of the catheter tip.

In a further embodiment of the present disclosure, the stretch section has an easily stretchable design. In this embodiment of the present disclosure, the easy stretchability of the stretch section is not necessarily achieved through a suitable choice of material, but by a corresponding design. The increase in length is not necessarily brought about by an elastic and/or plastic deformation, but by another change in shape of the stretch section, for example on account of an accordion movement. In principle, the stretch section can have any design suitable for the purpose of easy stretchability. In this embodiment of the present disclosure, the catheter shaft therefore has said easily stretchable design at least in the region of the stretch section, wherein the catheter shaft, away from the stretch section, has a different and less easily stretchable design, preferably a smooth-walled cylindrical design known from the prior art. If the stretch section takes up the entire length of the catheter shaft, then the entire catheter shaft has said easily stretchable design.

In a further embodiment of the present disclosure, the easily stretchable design is an accordion-like design. In this embodiment, the stretch section is designed in the manner of an accordion. This allows the stretch section to be easily stretched in the event of a corresponding tensile load. The accordion-like design can be achieved in particular by a folded or corrugated configuration of the stretch section, similar to a bendable drinking straw and/or a longitudinally elastic garden hose. In addition, a spiral-shaped design of the stretch section is conceivable and possible.

In a further embodiment of the present disclosure, the stretch section is made of an easily stretchable material. The easily stretchable material is preferably a plastic material. In this embodiment of the present disclosure, the catheter shaft is therefore made of said easily stretchable material at least in the region of the stretch section, wherein the catheter shaft, away from the stretch section, is made of a less easily stretchable material. If the stretch section takes up the entire length of the catheter shaft, then the entire catheter shaft is made of said easily stretchable material.

In a further embodiment of the present disclosure, the easily stretchable material comprises polyethylene and/or silicone. Alternatively, the easily stretchable material is made of polyethylene and/or silicone. The materials mentioned have proven to be particularly advantageous for the present use.

In a further embodiment of the present disclosure, the stretch section extends over an entire length of the catheter shaft. Consequently, in this embodiment of the present disclosure, the entire catheter shaft is easily stretchable. This embodiment of the present disclosure permits simplified production, since the catheter shaft is made of one and the same material and/or can have one and the same design. This is in contrast to embodiments in which the stretch section occupies only a part of the total length of the catheter shaft.

In a further embodiment of the present disclosure, the stretch section extends only over a part of the total length of the catheter shaft, and the catheter shaft, away from the stretch section, has a tensile compliance which is a maximum of 20%, preferably a maximum of 5%, particularly preferably a maximum of 2%, of the tensile compliance of the stretch section. To put it another way, the catheter shaft, away from the stretch section, has a tensile strength that is at least five times, preferably at least twenty times, particularly preferably at least fifty times, higher than the tensile strength of the stretch section. In this embodiment of the present disclosure, the stretch section is preferably arranged in the region of the distal end of the catheter shaft.

In a further embodiment of the present disclosure, the catheter shaft, away from the stretch section, has a different design and/or is made of a different material. The different design is less easily stretchable and preferably a smooth-walled cylinder. The different material is less easily stretchable and is preferably a plastic material commonly used for catheter shafts/catheter tubes and known to a person skilled in the art.

In a further embodiment of the present disclosure, the catheter further comprises a stretch-limiting device configured to limit the stretching of the stretch section. The stretch-limiting device can be used to limit stretching of the stretch section beyond a permissible dimension. The limitation on the stretching of the stretch section counteracts mechanical overloading of the stretch section and thus prevents the catheter shaft from tearing off. The stretch-limiting device is designed differently in different embodiments and can in principle have any form and function suitable for the present purpose.

In a further embodiment of the present disclosure, the stretch-limiting device comprises an elongate tension element bridging the stretch section to form a strain relief. The elongate tension element causes a mechanical bridging of the stretch section as soon as the latter reaches a maximum permissible stretching. The tension element forms said strain relief. The elongate tension element can be designed in any manner suitable for the present purpose.

In a further embodiment of the present disclosure, the elongate tension element comprises an electrically conductive wire configured for the transmission of electric current pulses for neurostimulation. The wire thus has an advantageous multiple function. Firstly, the wire acts as a strain relief. Secondly, the wire also acts as a conductor wire for said neurostimulation. In this embodiment of the present disclosure, the catheter shaft and/or the catheter tip has at least one stimulation electrode, which is electrically conductively contacted by means of the conductive wire or is formed by the wire.

In a further embodiment of the present disclosure, the catheter tip comprises a fixing device, which is configured for fixing the catheter tip in the proximal direction, in order to hinder a proximal dislocation of the catheter tip. The fixing device additionally counteracts said proximal dislocation. In other words, in the applied state of the catheter, the catheter tip is fixed by means of the fixing device in the region of the nerve that is to be anaesthetized. The fixing device can have any form and function suitable for the present purpose. For example, the catheter tip can have a specific design, which forms the fixing device. Alternatively or additionally, the catheter tip can be provided with fixing elements that counteract a proximal movement of the catheter tip. For this purpose, said fixing elements can be configured for frictional and/or positive-locking interaction with a body tissue surrounding the catheter tip.

The present disclosure also relates to a catheter assembly comprising a catheter according to the above description and comprising an insertion aid which extends between a distal end and a proximal end and has a lumen for insertion of the catheter shaft and has a connector, arranged at the proximal end of the insertion aid, for forming a releasable connection to the catheter hub. The insertion aid can also be called a capillary. The catheter hub is configured for releasable connection to the connector of the insertion aid and preferably has a complementary connector. Connectors suitable for the present purpose, for example Luer connectors, are known to those skilled in the art.

### BRIEF DESCRIPTION OF THE DRAWINGS

Further advantages and features of the present disclosure will emerge from the claims and from the following description of preferred exemplary embodiments of the present disclosure, which are illustrated with the aid of the drawings.

FIG. 1 shows a schematic side view of an embodiment of a catheter according to the present disclosure, having a catheter shaft with a stretch section;

FIG. 2 shows a further schematic side view of the catheter according to FIG. 1, wherein the stretch section assumes a stretched state;

FIGS. 3 and 4 show a schematic side view of an embodiment of a stretch section with an easily stretchable design, in a non-stretched state (FIG. 3) and in a stretched state (FIG. 4);

FIG. 5 shows a schematic side view of a catheter assembly having a catheter according to FIGS. 1 and 2 and an insertion aid, wherein the insertion aid is shown in longitudinal section;

FIG. 6 shows a further embodiment of a catheter according to the present disclosure in a schematic side view, wherein the stretch section takes up an entire length of the catheter shaft;

FIG. 7 shows a further embodiment of a catheter according to the present disclosure in a schematic side view, with a stretch-limiting device for limiting the stretching of the stretch section;

FIG. 8 shows a further embodiment of a catheter according to the present disclosure in a schematic side view, wherein the stretch-limiting device has a wire; and

FIG. 9 shows a further embodiment of a catheter according to the present disclosure in a schematic side view and with a fixing device.

### DETAILED DESCRIPTION

According to FIG. 1, a catheter 1 is provided for administering a local anaesthetic. In particular, the catheter 1 serves as a pain catheter in pain therapy, also referred to as peripheral nerve block.

The catheter 1 comprises a catheter shaft 2, a catheter tip 3 and a catheter hub 4.

The catheter shaft 2 extends between a proximal end 21 and a distal end 22.

The catheter tip 3 is arranged at the distal end 22 of the catheter shaft 2. In the embodiment shown, the catheter tip 3 is manufactured separately from the catheter shaft 2 and firmly connected to the distal end 22 thereof. Alternatively, the catheter tip 3 can be an integral part of the catheter shaft 2 and form the distal end 22 thereof.

The catheter hub 4 is arranged at the proximal end 21 of the catheter shaft 2. The catheter hub 4 is firmly connected to the proximal end 21.

It will be appreciated that the catheter 1 has at least one lumen extending through the catheter shaft 2 and the catheter hub 4, and at least one catheter outlet connected fluidically to the lumen for the purpose of dispensing the local anaesthetic. The at least one catheter outlet and the lumen are not shown in any detail here. The catheter outlet or the catheter outlets are preferably arranged in the region of the catheter tip. In this case, the at least one catheter outlet can extend axially through the catheter tip and/or radially through the catheter shaft. The number and arrangement of the catheter outlets is not essential as regards the present disclosure, and therefore further details are not explained.

For administration of the local anaesthetic, the catheter 1 is applied in a manner known to a person skilled in the art and is advanced distally until the catheter tip 3 is placed in immediate proximity to the nerve that is to be anaesthetized. The catheter 1 is preferably applied using an insertion aid, which will be explained in more detail below.

If, during the pain therapy, there is an unintended proximal movement of the catheter tip 3, referred to as (proximal) dislocation, the nerve to be anaesthetized is no longer sufficiently flushed by the local anaesthetic. This can adversely affect the efficacy of the pain therapy. To counteract this, the catheter shaft 2 comprises a stretch section 23.

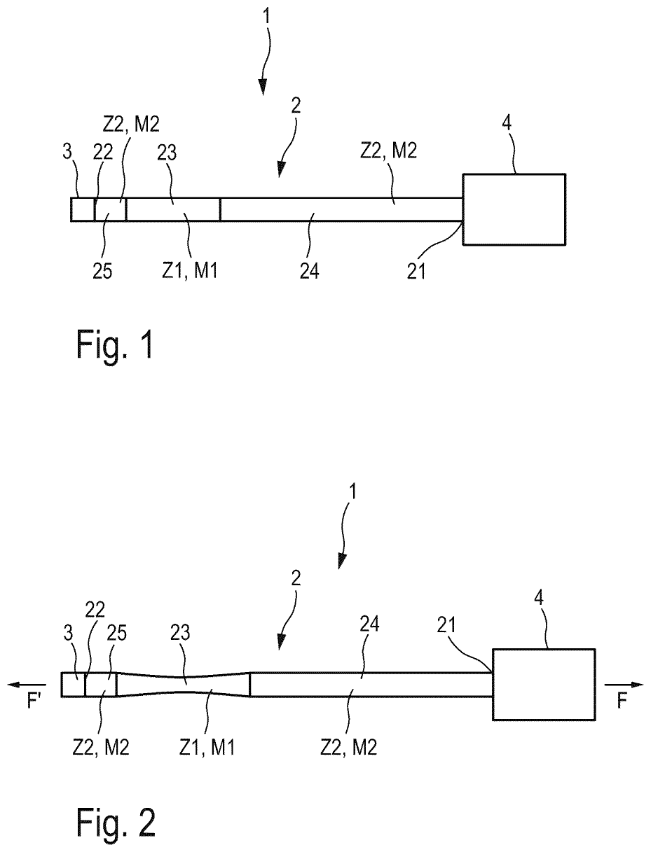

The stretch section 23 is configured to be easily stretchable, such that, in the event of proximal tensile loading F (see FIG. 2) of the catheter hub 4, an at least partial stretching of the catheter shaft 2 takes place. Thus, proximal dislocation of the catheter tip 3 can be avoided.

FIG. 2 shows the stretch section 23 in a stretched state. The stretched state of the stretch section 23 is caused by said proximal tensile loading F (and an opposite distal reaction force F′). For example, the proximal tensile loading F acts on the catheter hub 4 and the reaction force F′ acts on the catheter tip 3.

On account of the axial loading of the catheter shaft 2, the stretch section 23 is axially stretched and/or lengthened, in particular extended. This increase in length can be elastic, plastic or elastic-plastic depending on the specific design and material selection.

In the embodiment shown in FIGS. 1 and 2, the stretch section 23 covers only over a part of a total length of the catheter shaft 2. The arrangement of the stretch section 23 and its proportion of the total length are to be understood as examples.

In the present case, the stretch section 23 is arranged in the region of the distal end 22 of the catheter shaft 2. The catheter shaft 2 further comprises a proximal shaft section 24 and a distal shaft section 25. The proximal shaft section 24 is arranged axially between the proximal end 21 and the stretch section 23. The proximal shaft section 24 in the present case comprises the proximal end 21 of the catheter shaft 2 and is firmly connected at one end to the catheter hub 4. At the other end, the proximal shaft section 24 is firmly connected to the stretch section 23. The distal shaft section 25 is arranged axially between the catheter tip 3 and the stretch section 23. The distal shaft section 25 in the present case comprises the distal shaft end 22 and is firmly connected at one end to the catheter tip 3. At the other end, the distal shaft section 25 is firmly connected to the stretch section 23.

In an embodiment not shown in the figures, there is no distal shaft section 25, and instead the stretch section 23 is connected directly to the catheter tip 3.

The proximal shaft section 24 and the distal shaft section 25 are less easily stretchable than the stretch section 23. In other words, the stretch section 23 has a higher tensile compliance than the proximal shaft section 24 and the distal shaft section 25. To put it the other way round, the proximal shaft section 24 and the distal shaft section 25 have a higher tensile strength than the stretch section 23. The different design in terms of stretchability can be achieved through a correspondingly different material choice and/or design.

In the embodiment shown in FIGS. 1 and 2, the stretch section 23 has a tensile compliance Z1. Away from the stretch section 23, i.e. in the region of the proximal shaft section 24 and the distal shaft section 25, the catheter shaft 2 has a lower tensile compliance Z2. The two tensile compliances Z1, Z2 can also be referred to as first tensile compliance Z1 and second tensile compliance Z2.

The (first) tensile compliance Z1 of the stretch section is 100 mm/N in the present case. In embodiments not shown in the figures, the tensile compliance Z1 is at least 10 mm/N.

The (second) tensile compliance Z2 of the catheter shaft 2 away from the stretch section 23 is only 2% of the (first) tensile compliance Z1 in the present case. In embodiments not shown in the figures, the second tensile compliance is a maximum of 20%, preferably a maximum of 5%, of the first tensile compliance Z1.

In the embodiment according to FIGS. 1 and 2, the stretch section 23 is made of a material M1. Away from the stretch section 23, i.e. in the region of the proximal shaft section 24 and the distal shaft section 25, the catheter shaft 2 is made of a different material M2. The two materials M1, M2 can also be referred to as first material M1 and second material M2.

Both materials M1, M2 in the present case are plastic materials suitable for medical uses. The materials M1, M2 are different at least in terms of their tensile compliance.

In the present case, the first material M1 comprises polyethylene and/or silicone. In an embodiment not shown in the figures, the first material is polyethylene. In another embodiment not shown in the figures, the first material is silicone.

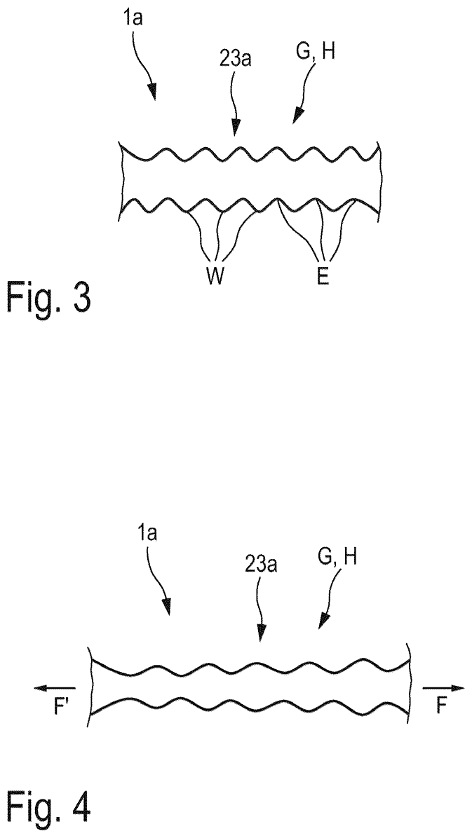

In an embodiment according to FIGS. 1 and 2, the easy stretchability of the stretch section 23 is thus achieved through a corresponding choice of material. Alternatively or additionally, the stretch section can have an easily stretchable design. Such an easily stretchable design is shown by way of example in FIGS. 3 and 4.

FIGS. 3 and 4 show an embodiment of a catheter 1a in which the stretch section 23a has an easily stretchable design G. In particular, the stretch section 23a is designed in the fashion of an accordion and in this sense has an accordion-like design H. The catheter shaft 2 is therefore provided, in the region of the stretch section 23, with radial protuberances W and radial indentations E. The protuberances W and the indentations E alternate in the axial direction and form said accordion-like design H.

FIG. 3 shows the stretch section 23a in a non-stretched state. FIG. 4 shows the stretch section 23a under a corresponding tensile load F, F′. The accordion-like design H is axially drawn/stretched in length under the influence of the tensile force F (and the reaction force F′).

It will be appreciated that the number, shape and dimensioning of the protuberances W and the indentations E shown in FIGS. 3 and 4 are to be understood purely as examples.

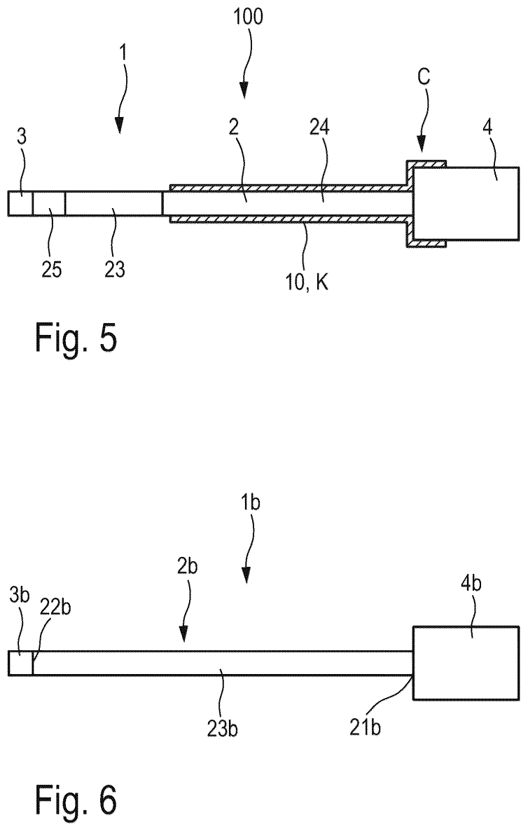

FIG. 5 shows a catheter assembly 100 with a catheter 1 according to FIGS. 1 and 2 and with an insertion aid 10. The insertion aid 10 is designed as a capillary K. The insertion aid 10 has a lumen (without reference sign), which is configured to receive the catheter shaft 2. In the configuration shown in FIG. 5, the catheter shaft 2 is pushed into said lumen via a proximal end (without reference sign) of the insertion aid 10, wherein the distal end of the catheter shaft 2 together with the catheter tip 3 and the stretch section 23 protrudes distally from the lumen of the insertion aid 10. The catheter hub 4 and the proximal end of the insertion aid are designed to form a releasable connection C. The releasable connection C can be a screw, Luer or other suitable connection for the present purpose.

FIGS. 6 to 9 show further embodiments of catheters 1b, 1c, 1d, 1e according to the present disclosure. In order to avoid repetition, the main differences between the catheters 1b, 1c, 1d, 1e and the catheter 1 and the catheter 1a according to FIGS. 1 to 4 are discussed below. Functionally identical components and/or sections of the catheters 1b, 1c, 1d, 1e are provided with identical reference numbers with the addition of lower-case letters. Unless stated otherwise, the details that have been disclosed concerning the catheters 1, 1a according to FIGS. 1 to 4 also apply mutatis mutandis to the catheters 1b, 1c, 1d, 1e according to FIGS. 6 to 9.

In the case of the catheter 1b according to FIG. 6, the stretch section 23b extends over an entire length of the catheter shaft 2b. Thus, in comparison to the catheter 1 according to FIGS. 1 and 2, simpler production can be achieved.

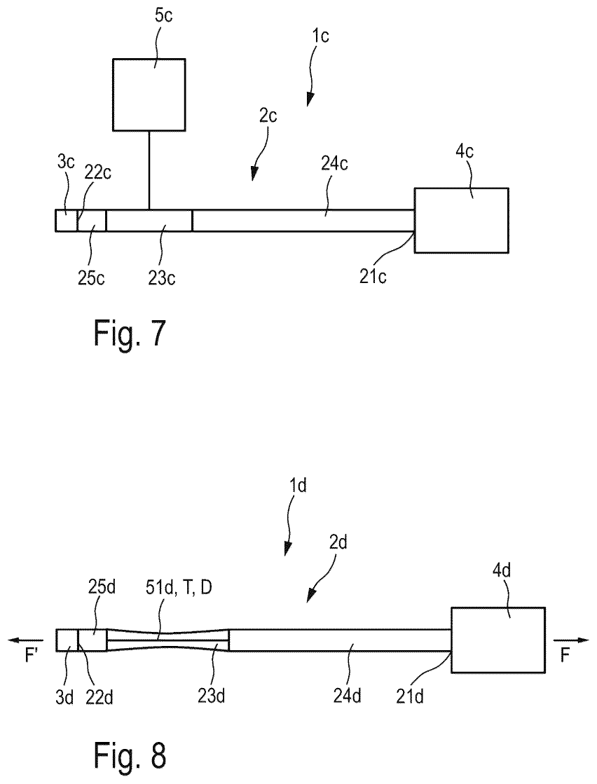

The catheter 1c according to FIG. 7 comprises a stretch-limiting device 5c, which is shown schematically in block illustration in FIG. 7. The stretch-limiting device 5c is assigned to the stretch section 23c. The stretch-limiting device 5c is configured to limit the stretching of the stretch section 23c. It is thus possible to counteract excessive stretching and associated mechanical overloading of the stretch section 23c. The stretch-limiting device 5c is designed differently in different embodiments. As soon as the stretch section 23c reaches a maximum permissible stretching, the stretch-limiting device preferably acts as a strain relief D and effects a force transmission between the proximal shaft section 24c and the distal shaft section 25c, bridging the stretch section 23c.

The catheter 1d according to FIG. 8 shows a variant of the catheter 1c, wherein the stretch-limiting device has an elongate tension element 51d. The elongate tension element bridges the (maximum permissibly stretched) stretch section 23d and forms said strain relief D.

In the embodiment shown in FIG. 8, the elongate tension element 51d comprises a conductive wire T, which forms said strain relief D. The conductive wire T extends along and/or in the stretch section 23d and is mechanically connected to the proximal shaft section 24d and the distal shaft section 25d. Alternatively or additionally, the conductive wire T can be mechanically connected to the catheter hub 4d and/or the catheter tip 3d. FIG. 8 shows only a portion of the conductive wire T.

The conductive wire T in the present case is designed on the one hand to form said strain relief D. On the other hand, the wire T is configured for the transmission and/or delivery of electric current pulses for neurostimulation.

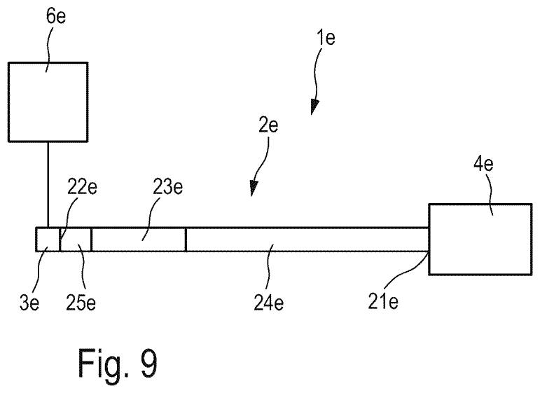

The catheter 1e according to FIG. 9 comprises a fixing device 6e, which is shown schematically in block illustration. The fixing device 6e is assigned to the catheter tip 3e. The fixing device 6e is configured for fixing the catheter tip 3e in the proximal direction, to hinder proximal dislocation of the catheter tip 3e. For this purpose, the fixing device 6e can have any suitable shape. For example, the fixing device 6e can have fixing elements which are formed on or attached to the catheter tip 3e and which are configured to interact with a body tissue surrounding the catheter tip 3e. By means of said interaction, the friction in the region of the catheter tip 3e can be increased or a positive fit with the body tissue can be achieved.

It will be appreciated that individual features of the catheters 1, 1a, 1b, 1c, 1d, 1e can be combined with one another to form further embodiments according to the present disclosure.

1. A catheter for administering a local anaesthetic, the catheter comprising:
a catheter shaft extending between a proximal end and a distal end;
a catheter hub arranged at the proximal end; and
a catheter tip arranged at the distal end,
the catheter shaft having a stretch section configured to be stretchable in such a way that an at least partial stretching of the catheter shaft occurs in response to a proximal tensile loading of the catheter hub, the at least partial stretching of the catheter shaft preventing a proximal dislocation or withdrawal of the catheter tip from an inserted position during a pain therapy.

2. The catheter according to claim 1, wherein the stretch section has a tensile compliance of at least 10 mm/N.

3. The catheter according to claim 1, wherein the stretch section has an accordion design.

4. The catheter according to claim 1, wherein the stretch section is made of an easily stretchable material.

5. The catheter according to claim 4, wherein the easily stretchable material comprises polyethylene and/or silicone.

6. The catheter according to claim 1, wherein the stretch section extends over an entire length of the catheter shaft.

7. The catheter according to claim 1, wherein:
the stretch section has a first tensile compliance,
the stretch section extends over only a portion of the entire length of the catheter shaft, and
the catheter shaft, away from the stretch section, has a second tensile compliance which is a maximum of 20% of the first tensile compliance.

8. The catheter according to claim 7, wherein the catheter shaft, away from the stretch section, has a different design and/or is made of a different material than the stretch section.

9. The catheter according to claim 1, further comprising a stretch-limiting device configured to limit stretching of the stretch section.

10. The catheter according to claim 9, wherein the stretch-limiting device comprises an elongate tension element bridging the stretch section to form a strain relief.

11. The catheter according to claim 10, wherein the elongate tension element comprises an electrically conductive wire configured for transmission and/or delivery of electrical current pulses for neurostimulation.

12. The catheter according to claim 1, wherein the catheter tip comprises a fixing device configured for fixing the catheter tip in a proximal direction to hinder a proximal dislocation of the catheter tip.

13. A catheter assembly comprising:
the catheter according to claim 1; and
an insertion aid extending between the distal end and the proximal end, the insertion aid having a lumen for insertion of the catheter shaft and a connector, arranged at the proximal end, for forming a releasable connection to the catheter hub.
